(19) United States
(12) Patent Application Publication
(10) Pub. No.: US 2026/0110811 A1
(43) Pub. Date: Apr. 23, 2026

(54) PET IMAGING SYSTEM WITH DEPTH-OF-INTERACTION INFORMATION EXTRACTION CAPACITIES AND RELATED METHOD

(71) Applicant: CANON MEDICAL SYSTEMS CORPORATION, Tochigi (JP)

(72) Inventors: Peng PENG, Vernon Hills, IL (US); Xiaoli LI, Vernon Hills, IL (US); Yi QIANG, Vernon Hills, IL (US)

(73) Assignee: CANON MEDICAL SYSTEMS CORPORATION, Tochigi (JP)

(21) Appl. No.: 18/924,048

(22) Filed: Oct. 23, 2024

(51) Int. Cl. G01T 1/29; A61B 6/03; G01T 1/20

(52) U.S. Cl. CPC G01T 1/2985; A61B 6/037; G01T 1/20184

(57) ABSTRACT

A PET apparatus is provided. The PET apparatus includes a scintillation array, a photosensor array, and processing circuitry. The scintillation array includes scintillator crystal units that are individually isolated with reflective material. Each scintillator crystal unit is configured to generate scintillation light in response to a gamma-ray interaction in the scintillator crystal unit that is caused by gamma-ray irradiation from an imaging object. Each scintillator crystal unit is configured with a substructure for decoding two or more depths within the scintillator crystal unit, such that gamma-ray interactions at different depths result in scintillation light that escapes from a light-escaping plane of the scintillator crystal unit with different patterns. The photosensor array is coupled to the scintillation array to convert the scintillation light received into electrical signals. The processing circuitry is configured to extract, from the electrical signals, information representing depth-of-interaction of the gamma-ray interactions in the scintillation array.

### BACKGROUND

### Field

This disclosure relates to positron emission tomography (PET) imaging systems.

### Description of the Related Art

Positron emission tomography (PET) is a functional imaging modality that is capable of imaging biochemical processes in humans or animals through the use of radioactive tracers. In PET imaging, a tracer agent is introduced into the patient to be imaged via injection, inhalation, or ingestion. After administration, the physical and bio-molecular properties of the agent cause it to concentrate at specific locations in the patient's body. The actual spatial distribution of the agent, the intensity of the region of accumulation of the agent, and the kinetics of the process from administration to its eventual elimination are all factors that may have clinical significance.

During this process, a tracer attached to the agent will emit positrons. When an emitted positron collides with an electron, an annihilation event occurs, wherein the positron and electron are combined. An annihilation event produces two gamma-ray photons (at 511 keV) traveling at substantially 180 degrees apart.

Time-of-flight (TOF) and depth-of-interaction (DOI) are two important metrics for evaluating the performance of a PET scanner. There is a preference to choose one over the other in different applications.

In clinical PET scanners, the emphasis is on TOF rather than DOI. TOF capabilities can improve the effective sensitivity of the system, which is critical for clinical applications. This improvement results in better image quality and lower radiation doses administrated to the patients. Due to the relatively large-bore diameter in clinical scanners, DOI assumes a less critical role.

Conversely, in preclinical PET scanners characterized by smaller-bore diameters, DOI information gains greater significance. By correcting parallax errors, a critical concern in preclinical applications, DOI capabilities play a pivotal role in enhancing spatial resolution.

It has been observed that DOI information can also contribute to enhancing TOF resolution. Moreover, with the increase in the axial length of clinical PET scanners, such as in total-body scanners, DOI becomes more important in correcting errors caused by highly oblique lines-of-response along the axial direction.

It is desirable to develop a scanner equipped with both good TOF resolution and DOI capabilities, so as to enhance the overall performance of clinical PET scanners.

### SUMMARY

The present disclosure relates to a positron emission tomography (PET) apparatus. The PET apparatus includes a scintillation array, a photosensor array, and processing circuitry. The scintillation array includes a plurality of scintillator crystal units that are individually isolated with reflective material. Each respective one of the plurality of scintillator crystal units is configured to generate scintillation light in response to a gamma-ray interaction in the scintillator crystal unit that is caused by gamma-ray irradiation from an imaging object. Each scintillator crystal unit is configured with a substructure for decoding two or more depths within the scintillator crystal unit, such that gamma-ray interactions at different depths result in scintillation light that escapes from a light-escaping plane of the scintillator crystal unit with different patterns. The photosensor array is coupled to the scintillation array to convert the scintillation light received from the scintillation array into electrical signals. The processing circuitry is configured to extract, from the electrical signals, information representing depth-of-interaction of the gamma-ray interactions in the scintillation array, and reconstruct, based on the extracted information, an image of the imaging object.

The disclosure additionally relates to a method for extracting DOI information in a PET imaging system. The method includes generating, via a scintillation array, scintillation light in response to gamma-ray interactions in the scintillation array that is caused by gamma-ray irradiation from an imaging object. The scintillation array includes a plurality of scintillator crystal units that are individually isolated with reflective material. Each scintillator crystal unit is configured with a substructure for decoding two or more depths within the scintillator crystal unit, such that gamma-ray interactions at different depths result in scintillation light that escapes from a light-escaping plane of the scintillator crystal unit with different patterns. The method also includes converting, via a photosensor array coupled to the scintillation array, the scintillation light received from the scintillation array into electrical signals. The method further includes extracting, from the electrical signals, information representing depth-of-interaction of the gamma-ray interactions in the scintillation array. The method further includes reconstructing, based on the extracted information, an image of the imaging object.

The disclosure additionally relates to a gamma-ray detector used in a PET imaging system. The gamma-ray detector includes a scintillation array including a plurality of scintillator crystal units that are individually isolated with reflective material. Each respective one of the plurality of scintillator crystal units is configured to generate scintillation light in response to a gamma-ray interaction in the scintillator crystal unit that is caused by gamma-ray irradiation from an imaging object. Each scintillator crystal unit is configured with a substructure for decoding two or more depths within the scintillator crystal unit, such that gamma-ray interactions at different depths result in scintillation light that escapes from a light-escaping plane of the scintillator crystal unit with different patterns.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIG. 1 shows a parallax error resulting from an oblique line-of-response (LOR) in a pair of positron emission tomography (PET) detector pixels without depth-of-interaction (DOI) capacities;

FIG. 2 shows a typical single-ended readout configuration in which a pixelated array of scintillator crystal units is coupled on the top of a pixelated array of photosensors;

FIG. 3 shows a modified single-ended readout configuration in accordance with embodiments of the disclosure, in which each scintillator crystal unit of the pixelated array has a substructure for decoding DOI information;

FIG. 4 shows an exemplary substructure design inside a scintillator crystal unit in accordance with embodiments of the disclosure;

FIG. 5A shows an exemplary substructure design inside a scintillator crystal unit in accordance with embodiments of the disclosure;

FIG. 5B shows an exemplary substructure design inside a scintillator crystal unit in accordance with embodiments of the disclosure;

FIGS. 6A and 6B show an exemplary scintillator crystal unit including four segments and the corresponding flood histogram formed by gamma-ray irradiation hitting the four segments, in accordance with embodiments of the disclosure;

FIGS. 7A and 7B show an exemplary scintillator crystal unit including eight segments and the corresponding flood histogram formed by gamma-ray irradiation hitting the eight segments, in accordance with embodiments of the disclosure;

FIG. 8 shows an exemplary scenario where a pixelized photosensor array is directly coupled to a pixelated array of scintillator crystal units in accordance with embodiments of the disclosure;

FIG. 9 shows an exemplary electronics design for obtaining timing, position, and energy information (time, x, y, energy) from the pixelized photosensor array, in accordance with embodiments of the disclosure;

FIG. 10 shows a flow chart of an exemplary procedure 1000 for extracting DOI information in accordance with embodiments of the disclosure;

FIG. 11A shows a perspective view of a PET scanner that can be used with the techniques described herein; and

FIG. 11B shows a schematic view of a PET scanner that can be used with the techniques described herein.

### DETAILED DESCRIPTION

The following disclosure provides embodiments or examples for implementing different features of the provided subject matter. Specific examples of components and arrangements are described below to simplify the present disclosure. These are, of course, merely examples and are not intended to be limiting.

For example, the order of discussion of the different steps as described herein has been presented for the sake of clarity. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Furthermore, as used herein, the words “a,” “an,” and the like generally carry a meaning of “one or more,”unless stated otherwise.

To achieve high sensitivity, positron emission tomography (PET) imaging scanners typically use thick detectors to give the required stopping power for 511 keV gamma photons. However, uncertainty in depth-of-interaction (DOI) information within these thick detectors can result in parallax errors. Such parallax degradation can become even worse at larger radial position within the PET field of view.

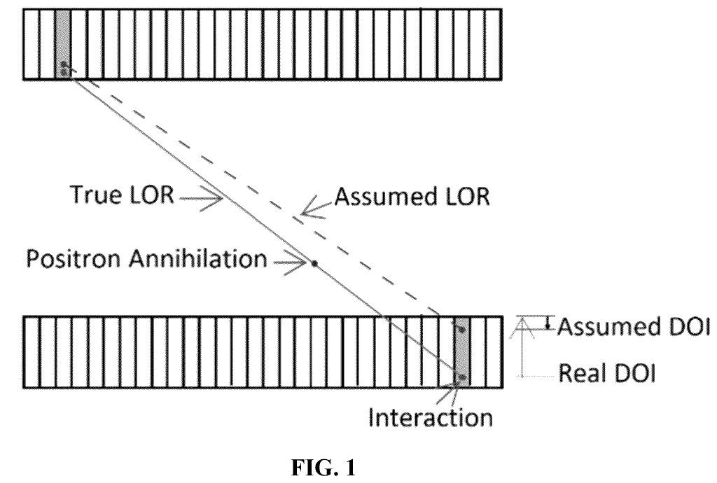

FIG. 1 shows a scenario where a parallax error arises from an oblique line-of-response (LOR) in PET detector pixels that lack DOI information. Without DOI capacities, gamma-ray interactions, which can occur over all depths within a detector pixel, are attributed to a single position. For example, the dashed line in FIG. 1 represents the assumed LOR, which is the same for all coincidence events between those two detector pixels. The solid line represents the true LOR, which can be exactly drawn if DOI information for that specific coincidence event is available. Such parallax errors can lead to artifacts and degradation in image quality.

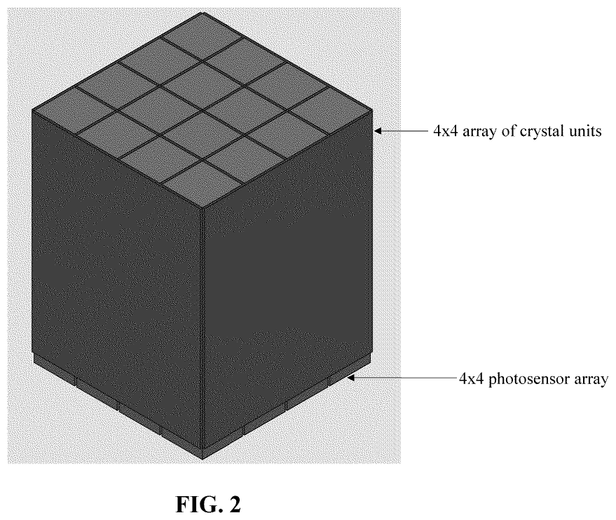

FIG. 2 shows a typical single-ended readout configuration. In this arrangement, a pixelated array of scintillator crystal units is coupled on the top of a pixelated array of photosensors. Specifically, the scintillator crystal units in the 4×4 crystal array are matched with the photosensors in the 4×4 photosensor array in a one-on-one manner. This configuration is optimal for time-of-flight (TOF) resolution, since individual photosensors can effectively capture most of the scintillation light emitted from the corresponding scintillator crystal units. However, a drawback of this design is the lack of DOI information, and it is a challenge to acquire DOI information without compromising TOF resolution or significantly increasing system costs.

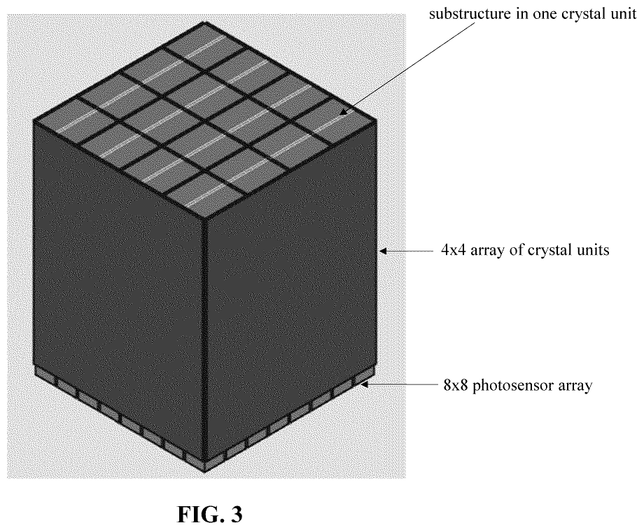

FIG. 3 shows an exemplary single-ended readout configuration in accordance with embodiments of the disclosure. As illustrated in FIG. 3, a 4×4 crystal array is coupled on the top of an 8×8 photosensor array. Each scintillator crystal unit in the pixelated array has a substructure designed to channel scintillation light generated in the scintillator crystal unit to distinct positions of the photosensor array. This design enables extraction of DOI information, as it can differentiate gamma-ray interactions occurring at different depths within individual scintillator crystal units.

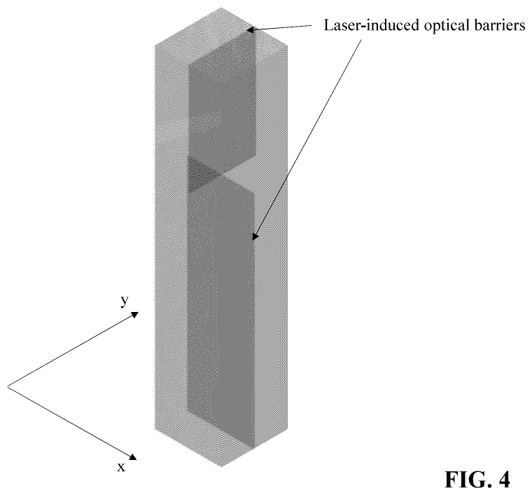

FIG. 4 shows an exemplary substructure design within a scintillator crystal unit in accordance with embodiments of the disclosure. This design includes using a high-power laser to engrave micro-cracks inside the crystal. The created micro-cracks form optical barriers that define light propagation pathways inside the crystal.

For instance, these laser-induced optical barriers (LIOBs) can be arranged as follows: in the top ⅓ of the scintillator crystal unit, the optical barrier plane is formed in the middle of the crystal unit along the y axis; in the bottom ⅔ of the crystal unit, the optical barrier plane is formed in the middle of the crystal unit along the x axis. This design leads to varying light distributions at the light-escaping plane (i.e., the bottom end surface of the crystal unit where the scintillation light is detected by the photosensors), for gamma-ray interactions at different depths within the crystal unit.

Note that the details shown in FIG. 4 are not restrictive. For example, the top optical barrier can be arranged at the top ⅔ of the crystal, while the bottom optical barrier can be at the bottom ⅓. Other divisions, fractions, and deployments can be used without departing from the spirit and scope of the disclosure.

Similarly, although the top and bottom optical barriers are shown as perpendicular to each other, it is possible to use angles other than 90 degrees between the optical barriers.

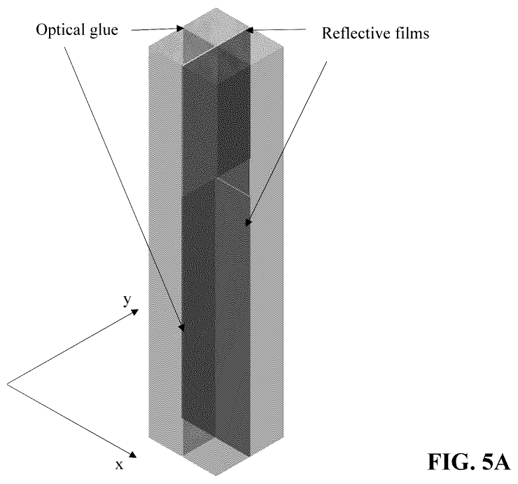

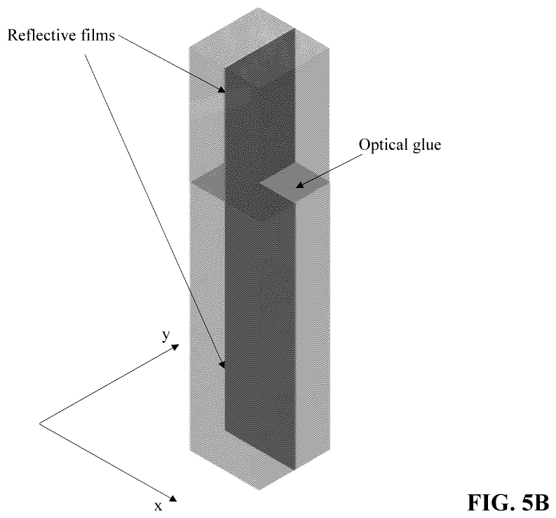

FIGS. 5A and 5B show two exemplary substructure designs within a scintillator crystal unit in accordance with embodiments of the disclosure. Both designs use four sub-crystals to form an individual crystal unit. In the top ⅓ of the crystal unit, reflective material (such as Enhanced Specular Reflector (ESR) films, BaSO₄ films, etc.) is applied in the middle plane along the y axis. In the bottom ⅔ of the crystal unit, reflective material is applied in the middle plane along the x axis. Optical glue is used on other contacting surfaces between the sub-crystals to allow light passage inside the crystal unit. The light pathways inside the scintillator crystal unit are thus defined by these contacting surfaces with reflective material and optical glue applied.

Similar to the example shown in FIG. 4, the details in FIGS. 5A and 5B are illustrative and not restrictive. One skilled in the art can recognize various modifications and variations applicable to the designs illustrated.

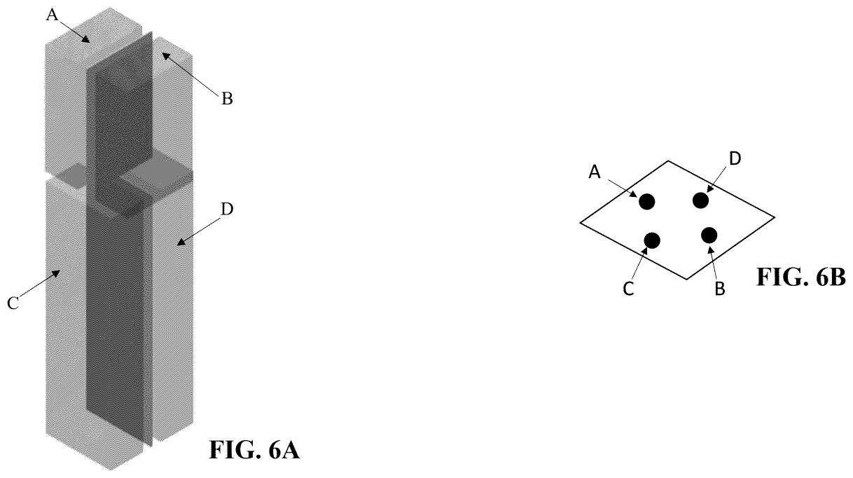

FIGS. 6A and 6B show an exemplary scintillator crystal unit including four segments and the corresponding flood histogram formed by gamma-ray irradiation hitting the four segments, in accordance with embodiments of the disclosure. The four segments can be implemented by the micro-cracks approach shown in FIG. 4, or by the sub-crystals approach shown in FIGS. 5A and 5B. Note that the distances between the segments do not indicate actual physical separations, but for better visual display of their positions in the drawing.

When gamma-ray irradiation hits different segments in FIG. 6A, the scintillation photons are distributed differently as they exit the scintillation crystal unit through the optical read-out surface (e.g., the bottom surface of the crystal unit). Four dots, A, B, C and D as shown in FIG. 6B, can be formed on the crystal flood histogram, corresponding to the four different gamma-ray interaction regions A, B, C, and D in the crystal unit, respectively. In this way, the depth-of-interaction information is encoded in the crystal flood histogram, and can be determined based on the crystal flood histogram.

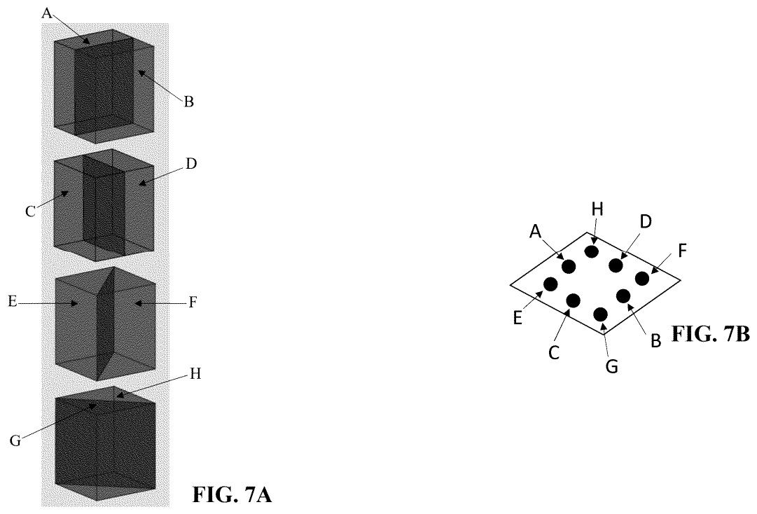

All the configurations shown in FIGS. 4, 5A, 5B, and 6A can decode two depth positions. Alternative crystal substructure designs allow the decoding of more than two depth positions. For instance, FIGS. 7A and 7B show a scintillator crystal unit including eight segments and the corresponding flood histogram formed by gamma-ray irradiation hitting the eight segments, in accordance with embodiments of the disclosure. Eight dots, A-H as shown in FIG. 7B, can be formed on the crystal flood histogram, corresponding to the eight different gamma-ray interaction regions A-H in the crystal unit, respectively. Based on the crystal flood histogram, it is possible to determine four different depths.

Furthermore, the scintillator crystal units can be isolated using highly reflective material. For example, each crystal unit can be wrapped with ESR films or BaSO₄ films. This approach ensures that the scintillation light intensity from each individual crystal unit is largely preserved, as in the PET detector design shown in FIG. 2, thereby leading to high TOF resolution. This is a distinct advantage compared with other designs where the amount of scintillation light on each individual photosensor may be compromised by light sharing or by the use of absorptive material in the crystal array.

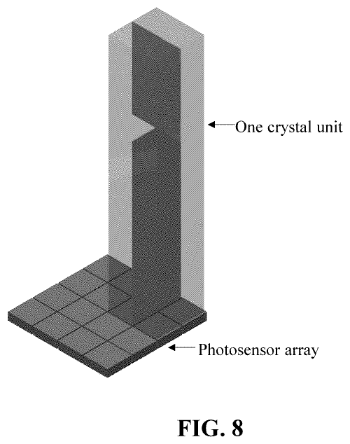

FIG. 8 shows an exemplary scenario where a pixelized photosensor array is directly coupled to a pixelized scintillator crystal array in accordance with embodiments of the disclosure. As each crystal unit in this arrangement is coupled with more than one photosensor to achieve DOI decoding, there is no need to arrange for a light guide between the crystal array and the photosensor array.

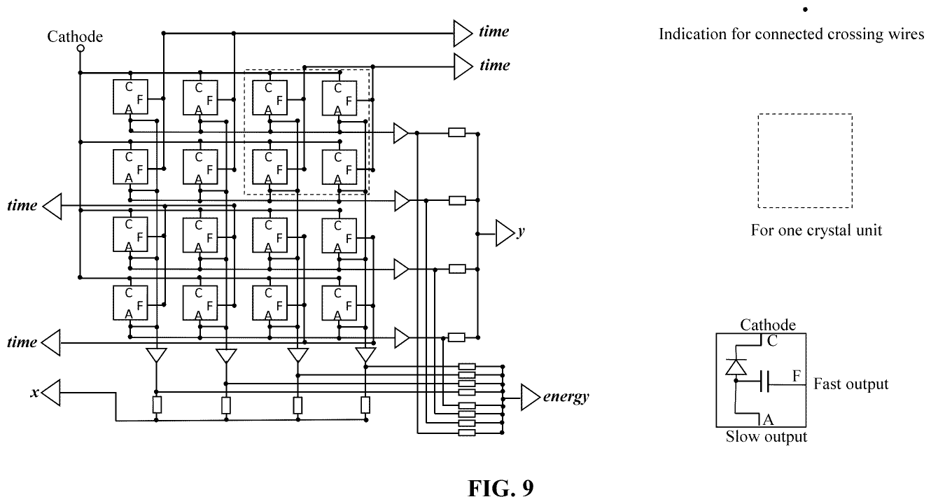

FIG. 9 shows an exemplary electronics design for obtaining timing, position, and energy information (t, x, y, e) from the pixelized photosensor array, in accordance with embodiments of the disclosure. As described above, when there is no light guide between the crystal array and the photosensor array, the scintillation light exiting each scintillator crystal unit is spread across a small portion of the photosensor array, e.g., the four photosensors underneath the crystal unit. The fast outputs of these four photosensors can be connected to one timing channel. For the case of a 4×4 photosensor array, there are four timing channels, as shown in FIG. 9. The slow component from the anode terminals of the four photosensors can be used to extract the position and energy information (x, y, e).

Each crystal unit generates its timing signal. In situations where multiple crystal units are hit by gamma-ray irradiation due to the Compton scatter effect, an energy-weighted mean can be used to calculate an averaged timing signal.

Various methods can be applied to decode the depth-of-interaction information from the crystal flood histogram. In one embodiment, the timing, position, and energy information acquired through the Anger logic electronics shown in FIG. 9 can be used to determine the following information of the gamma-ray interactions in the scintillation array: depth-of-interaction, crystal-of-interaction, energy-of-interaction, time-of-interaction. For example, the timing, position, and energy information can be inputted into a pre-trained neural network to extract the depth-of-interaction information. The crystal-of-interaction, energy-of-interaction, and/or time-of-interaction information can be obtained along with the depth-of-interaction information from the output of the neural network, for instance.

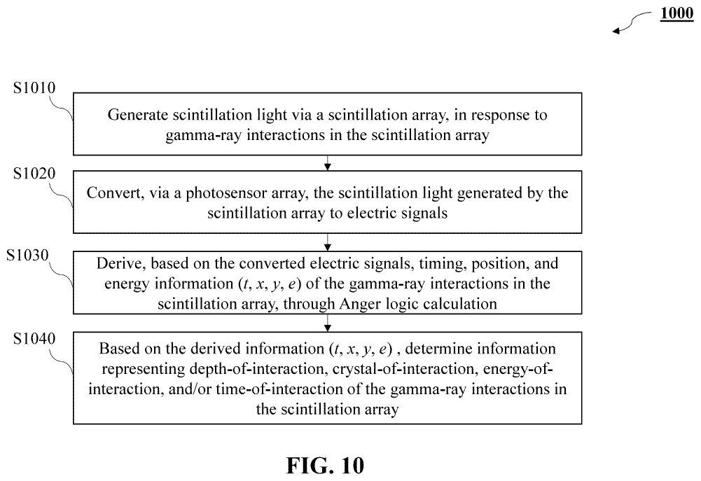

FIG. 10 shows a flow chart of an exemplary procedure 1000 for extracting DOI information in accordance with embodiments of the disclosure.

In step S1010, scintillation light is generated, via a scintillation array, in response to gamma-ray interactions in the scintillation array. In step S1020, the scintillation light generated by the scintillation array is converted to electric signals via a photosensor array. In step S1030, timing, position, and energy information (t, x, y, e) of the gamma-ray interactions in the scintillation array is derived through Anger logic calculation based on the converted electric signals. In step S1040, the derived information (t, x, y, e) can be used to determine the following information of the gamma-ray interactions in the scintillation array: depth-of-interaction, crystal-of-interaction, energy-of-interaction, time-of-interaction.

Subsequently, the extracted DOI information can be used in the image reconstruction process to enhance the image quality of the PET scanner by mitigating parallax errors. Additionally, by incorporating the extracted DOI information in the timing calibration process, the TOF resolution of the PET scanner also can be further improved.

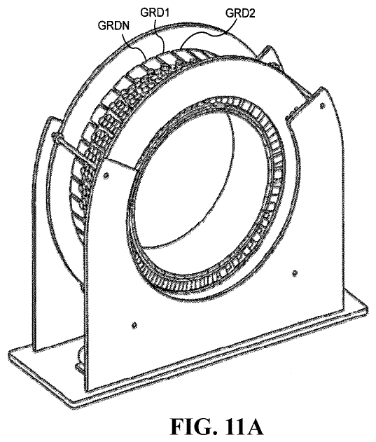

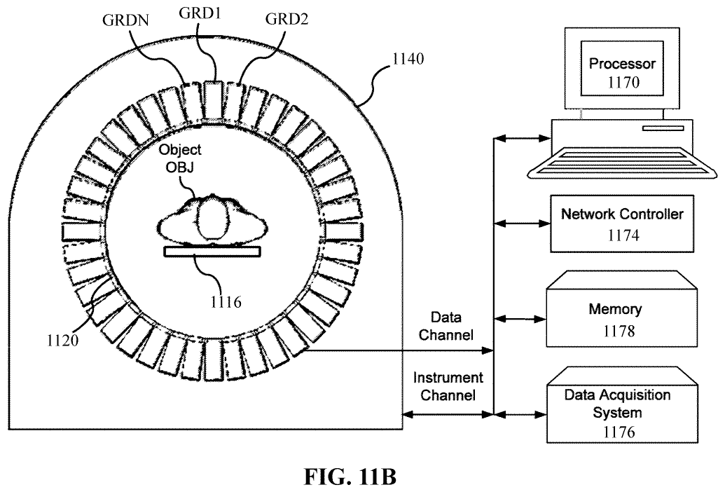

FIGS. 11A and 11B illustrate in implementation in which a medical imaging system includes a PET scanner that can implement the methods described in this disclosure. The PET scanner includes a plurality of gamma-ray detectors (GRDs) (e.g., GRD1, GRD2, through GRDN) that are each configured as rectangular detector modules.

Each GRD can include a two-dimensional array of individual detector crystals, which absorb gamma radiation and emit scintillation photons. The scintillation photons can be detected by a two-dimensional array of photodetectors or photosensors, e.g., photomultiplier tubes (PMTs), silicon photomultipliers (SiPMs), etc. A light guide can be disposed between the array of detector crystals and the photodetectors.

Each photodetector (e.g., PMT or SiPM) can produce an analog signal that indicates when scintillation events occur, and an energy of the gamma-ray producing the detection event. Moreover, the photons emitted from one detector crystal can be detected by more than one photodetector, and, based on the analog signal produced at each photodetector, the detector crystal corresponding to the detection event can be determined using Anger logic and crystal decoding, for example.

FIG. 11B shows one example of the arrangement of the PET scanner, in which the object OBJ to be imaged rests on a table 1116 and the GRD modules GRD1 through GRDN are arranged circumferentially around the object OBJ and the table 1116. The GRDs can be fixedly connected to a circular component 1120 that is fixedly connected to a gantry 1140. The gantry 1140 houses many parts of the PET scanner. The gantry 1140 of the PET scanner also includes an open aperture through which the object OBJ and the table 1116 can pass, and gamma-rays emitted in opposite directions from the object OBJ due to an annihilation event can be detected by the GRDs and timing and energy information can be used to determine coincidences for gamma-ray pairs.

In FIG. 11B, circuitry and hardware are also shown for acquiring, storing, processing, and distributing gamma-ray detection data. The circuitry and hardware include: a processor 1170, a network controller 1174, a memory 1178, and a data acquisition system (DAS) 1176. The PET scanner also includes a data channel that routes detection measurement results from the GRDs to the DAS 1176, the processor 1170, the memory 1178, and the network controller 1174. The data acquisition system 1176 can control the acquisition, digitization, and routing of the detection data from the detectors. In one implementation, the DAS 1176 controls the movement of the bed 1116. The processor 1170 performs functions including reconstructing images from the detection data, pre-reconstruction processing of the detection data, and post-reconstruction processing of the image data, as discussed herein.

The processor 1170 can be configured to perform various steps of the methods described herein and variations thereof. The processor 1170 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the processor 1170 can execute a computer program including a set of computer-readable instructions that perform various steps of the described methods, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xeon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

The memory 1178 can be a hard disk drive, CD-ROM drive, DVD drive, FLASH drive, RAM, ROM or any other electronic storage known in the art.

The network controller 1174, such as an Intel Ethernet PRO network interface card from Intel Corporation of America, can interface between the various parts of the PET scanner. Additionally, the network controller 1174 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G and 4G wireless cellular systems. The wireless network can also be WiFi, Bluetooth, or any other wireless form of communication that is known.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

Numerous modifications and variations of the embodiments presented herein are possible in light of the above teachings. It is therefore to be understood that within the scope of the claims, the application may be practiced otherwise than as specifically described herein. The inventions are not limited to the examples that have just been described; it is in particular possible to combine features of the illustrated examples with one another in variants that have not been illustrated.

Embodiments of the present disclosure may also be as set forth in the following parentheticals.

(1) A positron emission tomography (PET) apparatus, comprising: a scintillation array including a plurality of scintillator crystal units that are individually isolated with reflective material, each respective one of the plurality of scintillator crystal units being configured to generate scintillation light in response to a gamma-ray interaction in the scintillator crystal unit that is caused by gamma-ray irradiation from an imaging object, each scintillator crystal unit being configured with a substructure for decoding two or more depths within the scintillator crystal unit, such that gamma-ray interactions at different depths result in scintillation light that escapes from a light-escaping plane of the scintillator crystal unit with different patterns; a photosensor array coupled to the scintillation array to convert the scintillation light received from the scintillation array into electrical signals; and processing circuitry configured to extract, from the electrical signals, information representing depth-of-interaction of the gamma-ray interactions in the scintillation array, and reconstruct, based on the extracted information, an image of the imaging object.

(2) The apparatus of (1), wherein the substructure of each scintillator crystal unit includes a crystal bulk body with optical barriers arranged therein, the optical barriers being micro-cracks inside the crystal bulk body that are formed through a laser engraving process.

(3) The apparatus of (2), wherein in the substructure of each scintillator crystal unit, the optical barriers separate the crystal bulk body into four portions, such that gamma-ray interactions in the four portions result in scintillation light that escapes from the light-escaping plane of the scintillator crystal unit with four different patterns.

(4) The apparatus of (2), wherein in the substructure of each scintillator crystal unit, the optical barriers separate the crystal bulk body into eight portions, such that gamma-ray interactions in the eight portions result in scintillation light that escapes from the light-escaping plane of the scintillator crystal unit with eight different patterns.

(5) The apparatus of (1), wherein the substructure of each scintillator crystal unit includes a plurality of sub-crystals with reflective material and optical glue applied on different contacting interfaces between the plurality of sub-crystals.

(6) The apparatus of (5), wherein the substructure of each scintillator crystal unit includes four sub-crystals, such that gamma-ray interactions in the four sub-crystals result in scintillation light that escapes from the light-escaping plane of the scintillator crystal unit with four different patterns.

(7) The apparatus of (5), wherein the substructure of each scintillator crystal unit includes eight sub-crystals, such that gamma-ray interactions in the eight sub-crystals result in scintillation light that escapes from the light-escaping plane of the scintillator crystal unit with eight different patterns.

(8) The apparatus of (1), wherein the processing circuitry is further configured to perform, based on the electrical signals, processing to derive timing, position, and energy information (t, x, y, e) of the gamma-ray interactions in the scintillation array, and based on the derived information (t, x, y, e), determine the information representing depth-of-interaction of the gamma-ray interactions in the scintillation array.

(9) The apparatus of (8), wherein the processing circuitry is further configured to, based on the derived information (t, x, y, e), determine information representing crystal-of-interaction, energy-of-interaction, and time-of-interaction of the gamma-ray interactions in the scintillation array

(10) The apparatus of (1), wherein the photosensor array is configured with a finer level of granularity compared with the scintillation array.

(11) A method for extracting depth-of-interaction (DOI) information in a positron emission tomography (PET) imaging system, comprising: generating, via a scintillation array, scintillation light in response to gamma-ray interactions in the scintillation array that is caused by gamma-ray irradiation from an imaging object, the scintillation array including a plurality of scintillator crystal units that are individually isolated with reflective material, each scintillator crystal unit being configured with a substructure for decoding two or more depths within the scintillator crystal unit, such that gamma-ray interactions at different depths result in scintillation light that escapes from a light-escaping plane of the scintillator crystal unit with different patterns; converting, via a photosensor array coupled to the scintillation array, the scintillation light received from the scintillation array into electrical signals; extracting, from the electrical signals, information representing depth-of-interaction of the gamma-ray interactions in the scintillation array; and reconstruct, based on the extracted information, an image of the imaging object.

(12) The method of (11), wherein the substructure of each scintillator crystal unit includes a crystal bulk body with optical barriers arranged therein, the optical barriers being micro-cracks inside the crystal bulk body that are formed through a laser engraving process.

(13) The method of (12), wherein in the substructure of each scintillator crystal unit, the optical barriers separate the crystal bulk body into four portions, such that gamma-ray interactions in the four portions result in scintillation light that escapes from the light-escaping plane of the scintillator crystal unit with four different patterns.

(14) The method of (12), wherein in the substructure of each scintillator crystal unit, the optical barriers separate the crystal bulk body into eight portions, such that gamma-ray interactions in the eight portions result in scintillation light that escapes from the light-escaping plane of the scintillator crystal unit with eight different patterns.

(15) The apparatus of (11), wherein the substructure of each scintillator crystal unit includes a plurality of sub-crystals with reflective material and optical glue applied on different contacting interfaces between the plurality of sub-crystals.

(16) The method of (15), wherein the substructure of each scintillator crystal unit includes four sub-crystals, such that gamma-ray interactions in the four sub-crystals result in scintillation light that escapes from the light-escaping plane of the scintillator crystal unit with four different patterns.

(17) The method of (15), wherein the substructure of each scintillator crystal unit includes eight sub-crystals, such that gamma-ray interactions in the eight sub-crystals result in scintillation light that escapes from the light-escaping plane of the scintillator crystal unit with eight different patterns.

(18) The method of (11), wherein the extracting step further comprises: performing, based on the electrical signals, processing to derive timing, position, and energy information (t, x, y, e) of the gamma-ray interactions in the scintillation array, and based on the derived information (t, x, y, e), determining the information representing depth-of-interaction of the gamma-ray interactions in the scintillation array.

(19) The method of (18), wherein the determining step further comprises, based on the derived information (t, x, y, e), determining information representing crystal-of-interaction, energy-of-interaction, and time-of-interaction of the gamma-ray interactions in the scintillation array

(20) A gamma-ray detector used in a positron emission tomography (PET) imaging system, comprising: a scintillation array including a plurality of scintillator crystal units that are individually isolated with reflective material, each respective one of the plurality of scintillator crystal units being configured to generate scintillation light in response to a gamma-ray interaction in the scintillator crystal unit that is caused by gamma-ray irradiation from an imaging object, each scintillator crystal unit being configured with a substructure for decoding two or more depths within the scintillator crystal unit, such that gamma-ray interactions at different depths result in scintillation light that escapes from a light-escaping plane of the scintillator crystal unit with different patterns.

Numerous modifications and variations of the embodiments presented herein are possible in light of the above teachings. It is therefore to be understood that within the scope of the claims, the disclosure may be practiced otherwise than as specifically described herein.

What is claimed IS:

1. A positron emission tomography (PET) apparatus, comprising:
a scintillation array including a plurality of scintillator crystal units that are individually isolated with reflective material, each respective one of the plurality of scintillator crystal units being configured to generate scintillation light in response to a gamma-ray interaction in the scintillator crystal unit that is caused by gamma-ray irradiation from an imaging object, each scintillator crystal unit being configured with a substructure for decoding two or more depths within the scintillator crystal unit, such that gamma-ray interactions at different depths result in scintillation light that escapes from a light-escaping plane of the scintillator crystal unit with different patterns;
a photosensor array coupled to the scintillation array to convert the scintillation light received from the scintillation array into electrical signals; and
processing circuitry configured to
extract, from the electrical signals, information representing depth-of-interaction of the gamma-ray interactions in the scintillation array, and
reconstruct, based on the extracted information, an image of the imaging object.

2. The apparatus of claim 1, wherein the substructure of each scintillator crystal unit includes a crystal bulk body with optical barriers arranged therein, the optical barriers being micro-cracks inside the crystal bulk body that are formed through a laser engraving process.

3. The apparatus of claim 2, wherein in the substructure of each scintillator crystal unit, the optical barriers separate the crystal bulk body into four portions, such that gamma-ray interactions in the four portions result in scintillation light that escapes from the light-escaping plane of the scintillator crystal unit with four different patterns.

4. The apparatus of claim 2, wherein in the substructure of each scintillator crystal unit, the optical barriers separate the crystal bulk body into eight portions, such that gamma-ray interactions in the eight portions result in scintillation light that escapes from the light-escaping plane of the scintillator crystal unit with eight different patterns.

5. The apparatus of claim 1, wherein the substructure of each scintillator crystal unit includes a plurality of sub-crystals with reflective material and optical glue applied on different contacting interfaces between the plurality of sub-crystals.

6. The apparatus of claim 5, wherein the substructure of each scintillator crystal unit includes four sub-crystals, such that gamma-ray interactions in the four sub-crystals result in scintillation light that escapes the scintillator crystal unit with four different patterns.

7. The apparatus of claim 5, wherein the substructure of each scintillator crystal unit includes eight sub-crystals, such that gamma-ray interactions in the eight sub-crystals result in scintillation light that escapes from the light-escaping plane of the scintillator crystal unit with eight different patterns.

8. The apparatus of claim 1, wherein the processing circuitry is further configured to:
perform, based on the electrical signals, processing to derive timing, position, and energy information (t, x, y, e) of the gamma-ray interactions in the scintillation array, and
based on the derived information (t, x, y, e), determine the information representing depth-of-interaction of the gamma-ray interactions in the scintillation array.

9. The apparatus of claim 8, wherein the processing circuitry is further configured to, based on the derived information (t, x, y, e), determine information representing crystal-of-interaction, energy-of-interaction, and time-of-interaction of the gamma-ray interactions in the scintillation array.

10. The apparatus of claim 1, wherein the photosensor array is configured with a finer level of granularity compared with the scintillation array.

11. A method for extracting depth-of-interaction (DOI) information in a positron emission tomography (PET) imaging system, comprising:
generating, via a scintillation array, scintillation light in response to gamma-ray interactions in the scintillation array that is caused by gamma-ray irradiation from an imaging object, the scintillation array including a plurality of scintillator crystal units that are individually isolated with reflective material, each scintillator crystal unit being configured with a substructure for decoding two or more depths within the scintillator crystal unit, such that gamma-ray interactions at different depths result in scintillation light that escapes from a light-escaping plane of the scintillator crystal unit with different patterns;
converting, via a photosensor array coupled to the scintillation array, the scintillation light received from the scintillation array into electrical signals;
extracting, from the electrical signals, information representing depth-of-interaction of the gamma-ray interactions in the scintillation array; and
reconstruct, based on the extracted information, an image of the imaging object.

12. The method of claim 11, wherein the substructure of each scintillator crystal unit includes a crystal bulk body with optical barriers arranged therein, the optical barriers being micro-cracks inside the crystal bulk body that are formed through a laser engraving process.

13. The method of claim 12, wherein in the substructure of each scintillator crystal unit, the optical barriers separate the crystal bulk body into four portions, such that gamma-ray interactions in the four portions result in scintillation light that escapes from the light-escaping plane of the scintillator crystal unit with four different patterns.

14. The method of claim 12, wherein in the substructure of each scintillator crystal unit, the optical barriers separate the crystal bulk body into eight portions, such that gamma-ray interactions in the eight portions result in scintillation light that escapes from the light-escaping plane of the scintillator crystal unit with eight different patterns.

15. The apparatus of claim 11, wherein the substructure of each scintillator crystal unit includes a plurality of sub-crystals with reflective material and optical glue applied on different contacting interfaces between the plurality of sub-crystals.

16. The method of claim 15, wherein the substructure of each scintillator crystal unit includes four sub-crystals, such that gamma-ray interactions in the four sub-crystals result in scintillation light that escapes from the light-escaping plane of the scintillator crystal unit with four different patterns.

17. The method of claim 15, wherein the substructure of each scintillator crystal unit includes eight sub-crystals, such that gamma-ray interactions in the eight sub-crystals result in scintillation light that escapes from the light-escaping plane of the scintillator crystal unit with eight different patterns.

18. The method of claim 11, wherein the extracting step further comprises:
performing, based on the electrical signals, processing to derive timing, position, and energy information (t, x, y, e) of the gamma-ray interactions in the scintillation array, and
based on the derived information (t, x, y, e), determining the information representing depth-of-interaction of the gamma-ray interactions in the scintillation array.

19. The method of claim 18, wherein the determining step further comprises, based on the derived information (t, x, y, e), determining information representing crystal-of-interaction, energy-of-interaction, and time-of-interaction of the gamma-ray interactions in the scintillation array.

20. A gamma-ray detector used in a positron emission tomography (PET) imaging system, comprising:
a scintillation array including a plurality of scintillator crystal units that are individually isolated with reflective material, each respective one of the plurality of scintillator crystal units being configured to generate scintillation light in response to a gamma-ray interaction in the scintillator crystal unit that is caused by gamma-ray irradiation from an imaging object, each scintillator crystal unit being configured with a substructure for decoding two or more depths within the scintillator crystal unit, such that gamma-ray interactions at different depths result in scintillation light that escapes from a light-escaping plane of the scintillator crystal unit with different patterns.
